(19) United States
(12) Patent Application Publication
(10) Pub. No.: US 2018/0207312 A1
(43) Pub. Date: Jul. 26, 2018

(54) Filtering Apparatus

(71) Applicant: Fancy Food Service Equipment Co., Ltd., Kaohsiung City (TW)

(72) Inventor: Tsung-Che Chiang, Kaohsiung City (TW)

(21) Appl. No.: 15/413,659

(22) Filed: Jan. 24, 2017

(51) Int. Cl. A61L 9/22; B01D 46/00; B01D 46/24; B01D 46/42; F24C 15/20; A61L 9/16

(52) U.S. Cl. CPC A61L 9/22; B01D 46/0028; B01D 46/0038; A61L 2209/14; B01D 46/4245; F24C 15/2042; A61L 9/16; B01D 46/2418

(57) ABSTRACT

A filtering apparatus includes a filter with meshes having a periphery enclosed by a metal frame, an antibacterial material layer formed on the filter, a negative ions generator stretching into the filter for generating negative oxygen ions, and an electrode plate disposed on the metal frame. The metal frame is affected by the electrode plate to produce a high voltage electric field, thereby activating the antibacterial material layer under a piezoelectric effect to release more negative oxygen ions and silver ions. The negative oxygen ions and the silver ions collide together within the filter and become finer, thereby absorbing smelly odor passing through the filter to the maximum extent, purifying air, destroying germs, and promoting the air quality of the environment.

### BACKGROUND OF THE INVENTION

### 1. Field of the Invention

This invention relates to an air purifying apparatus and relates particularly to a filtering apparatus capable of absorbing smelly odor, cleaning air, and disinfecting germs effectively.

### 2. Description of the Related Art

Generally, it is known that the air quality is linked to human health closely. Air with good quality can increase oxygen content of blood within human body, purify blood, activate human cells, and accelerate human metabolism after being breathed into human body. Accordingly, air is one of the essential elements to prolong human lifespan. However, owing to the social prosperity and the progress of science and technology, an amount of air pollutants is increased to further worsen the air quality. The air pollutants are mainly resulted from black smoke produced by burning fuels and dust discharged by industrial manufacturing processes, construction works, and vehicles. The air pollutants can be divided by particle size into visible dust and invisible particulate matters. The invisible particulate matters suspended in the air are harmful to human health. For instance, the particulate matters may follow the air to be breathed into human body to further harm and infect human respiratory tract. Meanwhile, the particulate matters may cause people who have sensitive constitutions to have symptoms like dry eyes, dry or sore throat, coughing, sneezing, allergy reaction, headache, even lethargy, distraction and so on. Hence, the air quality can affect human health greatly.

Although many countries become conscious of serious influences resulted from the bad air quality and establish relevant standards for use in improving the air quality by restraining the amount of discharged air pollutants of each countries from going beyond the limits, effects are unremarkable. Since it is barely possible to improve the air quality within a short time, people start to have a craze with buying and installing air cleaners in their houses or offices in order to purify indoor air and further prevent their health from injuries by the hurtful outdoor air quality. However, the air cleaners can only filter dust and coarse particulate matters that have larger particle sizes without absorbing smelly odor in the air. Further, the air cleaners are of no use to percolate cooking fume. Thus, the applicant advances a filtering apparatus aiming at improving aforementioned deficiencies.

### SUMMARY OF THE INVENTION

The object of this invention is to provide a filtering apparatus capable of absorbing smelly odor, purifying air, and sterilizing germs in order to better the air quality of the environment effectively.

The filtering apparatus of this invention includes a filter with several meshes having a periphery enclosed by a metal frame, an antibacterial material layer covering the filter, the antibacterial material layer containing silver content and being capable of generating negative oxygen ions, an electrode plate fitted on the metal frame, and a negative ions generator fitted on the metal frame and reaching into the filter for producing the negative oxygen ions. Hence, when cooking fume and smelly odor pass through the filter, the antibacterial material layer can take particulate matters and smelly odor in the air. Meanwhile, the metal frame is electrically connected to the electrode plate to produce a high voltage electric field in order that the antibacterial material layer is affected under a piezoelectric effect. Therefore, the antibacterial material layer covering the filter can release more negative oxygen ions and silver ions. Further, the negative oxygen ions and silver ions crack together within the filter to allow the negative oxygen ions and the silver ions to be finer. Hence, a large number of the negative oxygen ions can cover the smelly odor completely and the finer negative oxygen ions and silver ions can permeate into the smelly odor, particulate matters, and cooking fume to sanitize and inhibit germs, clean the air, and improve the air quality. Simultaneously, the air processed by the filtering apparatus can be discharged to inside and outside of a building directly without harming human health, thereby increasing the air quality of the environment.

Preferably, the meshes of the filter are formed into a shape of honeycomb.

Preferably, the antibacterial material layer is an antibacterial material stuck to the filter after the filter is immersed in the antibacterial material.

Preferably, the filter is made from polycarbonate hollow fibers.

Preferably, the metal frame is made of copper.

Preferably, the antibacterial material layer is an antibacterial material, components of which have nano-sized tourmaline, nano-sized silver impregnated activated carbon, nano-sized zeolite antibacterial, nano-sized titania, and nano-sized dispersant.

Preferably, the nano-sized tourmaline is from 5 to 10 percent by weight, the nano-sized silver impregnated active carbon is from 60 to 75 percent by weight, the nano-sized zeolite antibacterial is from 10 to 15 percent by weight, the nano-sized titania is from 5 to 10 percent by weight, and the nano-sized dispersant is from 3 to 5 percent by weight.

Preferably, the filtering apparatus is further installed within a teppanyaki assembly. The teppanyaki assembly has a base, a cooking plate fitted on the base, at least one intake opening fitted at one side of the cooking plate, an exhaust unit fitted within the base, at least one main discharge opening connected with the exhaust unit, and an auxiliary discharge opening fitted on another side of the cooking plate and corresponding to the intake opening. The exhaust unit has an exhaust blower fitted in the base, a first discharge pipe connected between the exhaust blower and the intake opening, a second discharge pipe connected between the exhaust blower and the main discharge opening, and an intake pipe connected between the auxiliary discharge opening and the exhaust blower. The filtering apparatus is fitted between the first discharge pipe and the exhaust blower.

### BRIEF DESCRIPTION OF THE DRAWINGS

FIG. 1 is a schematic view showing a first preferred embodiment of this invention; and

FIG. 2 is a schematic view showing a simulation that the filtering apparatus is positioned within a teppanyaki assembly.

### DETAILED DESCRIPTION OF THE PREFERRED EMBODIMENTS

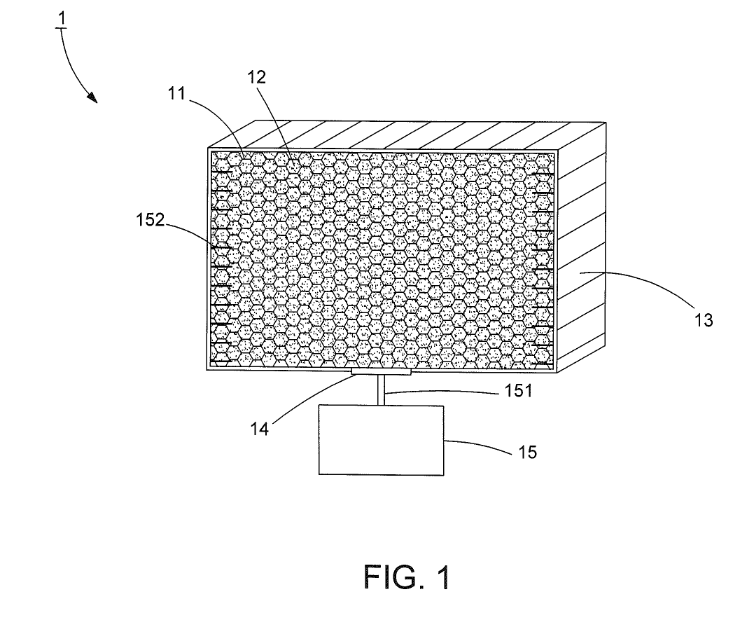

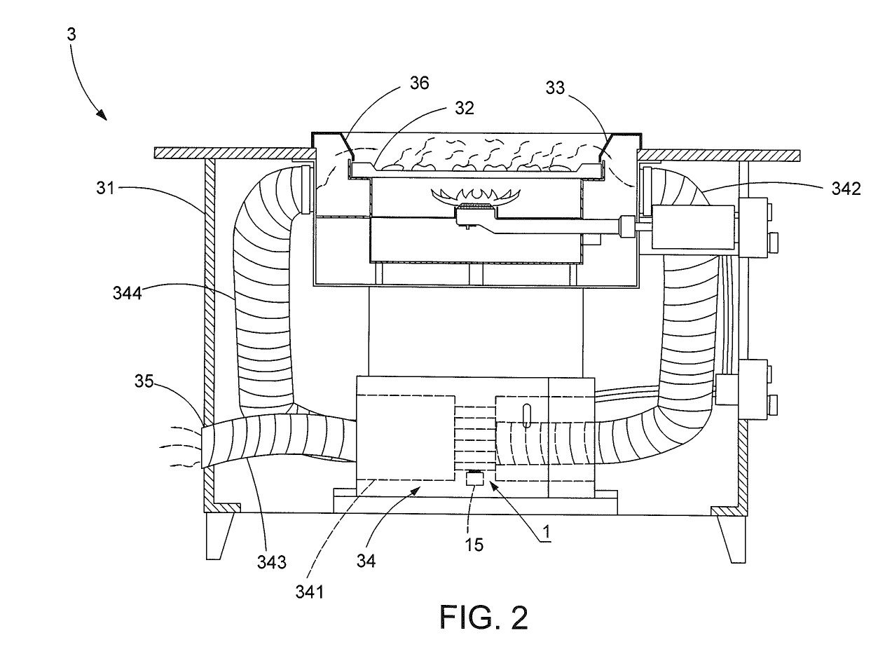

Referring to FIG. 1, a first preferred embodiment of a filtering apparatus 1 of this invention can be applied to an assembly aimed at clarifying air, such as air cleaners (not shown) for use in indoor spaces or teppanyaki assemblies 3 as shown in FIG. 2 for use in business sites. Here takes that the filtering apparatus 1 of this invention is installed within a teppanyaki assembly 3 as an example. The filtering apparatus 1 includes a filter 11 provided with several meshes formed thereon, an antibacterial material layer 12 shaped on the filter 11, a metal frame 13 encircling a periphery of the filter 11, an electrode plate 14 fitted on the metal frame 13, and a negative ions generator 15 fitted on the metal frame 13 and elongating into the filter 11. The antibacterial material layer involves silver content and is capable of producing negative oxygen ions. Here take that the meshes of the filter 11 are provided with a honeycombed arrangement and made from polycarbonate hollow fibers as examples.

In this preferred embodiment, the metal frame 13 covering the periphery of the filter 11 is made of copper. Meanwhile, the antibacterial material layer 12 is an antibacterial material (not shown) attached to the filter 11 after the filter 11 is dipped into the antibacterial material so that the antibacterial material can permeate the whole meshes of the filter 11 fully. Components of the antibacterial material include nano-sized tourmaline occupying 5 to 10 percent by weight, nano-sized silver impregnated active carbon occupying 60 to 75 percent by weight, nano-sized zeolite antibacterial occupying 10 to 15 percent by weight, nano-sized titania occupying 5 to10 percent by weight, and nano-sized dispersant occupying 3 to 5 percent by weight. The tourmaline which is one kind of borosilicate minerals with autogenous electrification has an asymmetric crystal structure. One side of the crystal structure is the positive electrode and another side is the negative electrode. Thus, electrons within the tourmaline flow from the negative electrode to the positive electrode ceaselessly to form electric currents and electrostatic fields. Meanwhile, the ability of releasing negative oxygen ions from the tourmaline has close links with a size of the crystal structure of the tourmaline. In other words, when the tourmaline is crushed, the release ability of crushed tourmaline is more enhanced to release more negative oxygen ions if the crushed size of the tourmaline, preferably nano-sized, is closer to a size of the single crystal structure. Further, a piezoelectric effect of the tourmaline is more enhanced when the crystal structure of the tourmaline is heated and pressurized. Simultaneously, the ability of releasing negative oxygen ions from the crushed tourmaline can be increased, e.g. up to ten times or more than ten times. Therefore, the addition of the dispersant can prevent the self-assembly of the nano-sized tourmaline and allow a preferable releasing effect of the negative oxygen ions from the tourmaline. Besides, the silver impregnated active carbon is one kind of minerals capable of releasing silver ions which can be used for damaging germs. Moreover, the zeolite antibacterial is one kind of porous minerals which has the preferable thermal stability, antibacterial and bacteriostatic effects.

The negative ions generator 15 has an electrically conductive pin 151 connected with the electrode plate 14 and at least one discharge unit 152 stretching into the filter 11 for releasing the negative oxygen ions. The electrically conductive pin 151 is provided with resilience. Therefore, when the negative ions generator 15 operates to release the negative oxygen ions, the electrically conductive pin 151 is driven to form a high voltage electric field on the metal frame 13 which is made of a metal material. The tourmaline of the antibacterial material layer 12 is activated by the high voltage electric field to release more negative oxygen ions and silver ions under the piezoelectric effect. Further, the negative oxygen ions and the silver ions bump with each other within the filter 11 in order to refine the negative oxygen ions and silver ions. Accordingly, not only the negative ions generator 15 can release the negative oxygen ions but the tourmaline is activated to release the negative oxygen ions under piezoelectric effect the within the filter 11, thereby fulfilling the filter 11 with a great number of the refined negative oxygen ions.

Referring to FIG. 2 shows that the filtering apparatus 1 of this invention is positioned within the teppanyaki assembly 3. The teppanyaki assembly includes a base 31, a cooking plate 32 fitted on the base 31, at least one intake opening 33 located at one side of the cooking plate 32, an exhaust unit 34 fitted within the base 31, at least one main discharge opening 35 connected with the exhaust unit 34, and an auxiliary discharge opening 36 located at another side of the cooking plate 32 and opposite to the intake opening 33. The exhaust unit 34 has an exhaust blower 341 fitted in the base 31, a first discharge pipe 342 connected between the exhaust blower 341 and the intake opening 33, a second discharge pipe 343 connected between the exhaust blower 341 and the main discharge opening 35, and an intake pipe 344 connected between the auxiliary discharge opening 36 and the exhaust blower 341. The filtering apparatus 1 is mounted between the first discharge pipe 342 and the exhaust blower 341. After the cooking fume is involved and filtered by the filtering apparatus 1, the purified air can be discharged to indoor space through the main discharge opening 35, thereby facilitating the circulation of indoor air and reducing the waste of energy resources of air conditioning devices. Meanwhile, the purified air can also be discharged through the auxiliary discharge opening 36 toward a direction of the cooking plate 32 in order to blow the cooking fume toward a direction of the intake opening 33. Hence, the cooking fume can be sucked from the intake opening 33 and further clarified by the filtering apparatus 1 through the first discharge pipe 342.

Referring to FIGS. 1 and 2, when cooking ingredients are cooked on the cooking plate 32, the exhaust unit 34 fitted within the base 31 is functioning simultaneously. Thus, the cooking fume which is produced when the cooking ingredients are heated by the cooking plate 32 can be absorbed by the exhaust unit 34. Because the auxiliary discharge opening 36 is located at a place corresponding to the intake opening 33, the purified air discharged from the auxiliary opening 36 can blow the cooking fume which is floated on the cooking plate 32 toward the direction of the intake opening 33. Meanwhile, the exhaust blower 341 operates to produce an absorbing effect at the intake opening 33. Therefore, the cooking fume can be affected by the absorbing effect of the exhaust blower 341 to be pushed toward a direction of the filtering apparatus 1 through the first discharge pipe 342 effectively. Then, the negative ions generator 15 fitted on the metal frame 13 is driven to release the negative oxygen ions from the discharge unit 152 and further produce the high voltage electric field on the metal frame 13 by the electrically conductive pin 151 in order to activate the tourmaline of the antibacterial material layer 12 to form the piezoelectric effect. Hence, the tourmaline and the silver impregnated active carbon of the antibacterial material layer 12 are stimulated to release more negative oxygen ions and silver ions within the filter 11. Further, the negative oxygen ions and silver ions smash with each other to refine the negative oxygen ions and silver ions. Whereby the meshes of the filter 11 are filled with the negative oxygen ions and the refined negative oxygen ions and silver ions. Accordingly, when the cooking fume and smelly odor float through the meshes of the filter 11, the cooking fume and the smelly odor can be involved by the great number of the negative oxygen ions effectively. Meanwhile, the finer smelly odor can be clarified by the refined negative oxygen ions and silver ions and the germs can be inhabited by the zeolite antibacterial to provide the filtered, sterilized and purified air. The purified air is then discharged from the main discharge opening 35 of the second discharge pipe 343 and the auxiliary discharge opening 36 of the intake pipe 344 toward the outside. Thus, the cooking fume and the smelly odor can be strained, disinfected and cleaned effectively by the filtering apparatus 1. Meanwhile, the cooling plate 32 can cause the circulation of air, so the cooking fume can only flow among the cooking plate 32 to prevent clothes of customers from being stained by the cooking fume and to promote the effect of filtering, purifying and sterilizing the cooking fume. Therefore, the air quality of the environment can be largely improved.

To sum up, the filtering apparatus of this invention takes advantages of the filter, the antibacterial material layer, the metal frame surrounding the periphery of the filter, the electrode plate, and the negative ions generator to purify the air. The negative ions generator and the electrode plate which is disposed on the metal frame activate the metal frame to form the high voltage electric field to further activate the antibacterial material layer to release a large number of the negative oxygen ions and silver ions under the piezoelectric effect. Meanwhile, the negative oxygen ions and silver ions within the filter bump together to be refined. The refined negative oxygen ions and silver ions can absorb the cooking fume, smelly odor and particulate matters preferably, thereby filtering and purifying the air and further bettering the air quality of the environment effectively.

What is claimed is:

1. A filtering apparatus comprising a filter with a plurality of meshes formed thereon, an antibacterial material layer formed on said filter and provided with silver content and production of negative oxygen ions, a metal frame surrounding a periphery of said filter, an electrode plate disposed on said metal frame, and a negative ions generator disposed on said metal frame and extended into said filter, wherein said negative ions generator produces and releases negative oxygen ions, said metal frame being activated by said electrode plate to form a high voltage electric field whereby said antibacterial material layer releases more negative oxygen ions under a piezoelectric effect, said negative oxygen ions entering said plurality of meshes of said filter and colliding with each other to refine said negative oxygen ions and to facilitate an increased purifying effect of said negative oxygen ions.

2. The filtering apparatus as claimed in claim 1, wherein said meshes of said filter are in a honeycombed arrangement.

3. The filtering apparatus as claimed in claim 1, wherein said antibacterial material layer is an antibacterial material adhered to said filter after said filter is impregnated with said antibacterial material.

4. The filtering apparatus as claimed in claim 1, wherein said filter is made from polycarbonate hollow fibers.

5. The filtering apparatus as claimed in claim 1, wherein said metal frame is made of copper.

6. The filtering apparatus as claimed in claim 1, wherein said antibacterial material layer is an antibacterial material, components of which include nano-sized tourmaline, nano-sized silver impregnated activated carbon, nano-sized zeolite antibacterial, nano-sized titania, and nano-sized dispersant.

7. The filtering apparatus as claimed in claim 6, wherein said nano-sized tourmaline is from 5 to10 percent by weight, said nano-sized silver impregnated active carbon is from 60 to 75 percent by weight, said nano-sized zeolite antibacterial is from 10 to 15 percent by weight, said nano-sized titania is from 5 to10 percent by weight, and said nano-sized dispersant is from 3 to 5 percent by weight.

8. The filtering apparatus of claim 1 being further mounted within a teppanyaki assembly, said teppanyaki assembly having a base, a cooking plate disposed on said base, at least one intake opening disposed at one side of said cooking plate, an exhaust unit disposed within said base, at least one main discharge opening connected with said exhaust unit, and an auxiliary discharge opening disposed on another side of said cooking plate and opposite to said intake opening, said exhaust unit having an exhaust blower disposed in said base, a first discharge pipe connected between said exhaust blower and said intake opening, a second discharge pipe connected between said exhaust blower and said main discharge opening, and an intake pipe connected between said auxiliary discharge opening and said exhaust blower, said filtering apparatus being disposed between said first discharge pipe and said exhaust blower.
